(19) United States
(12) Patent Application Publication
(10) Pub. No.: US 2016/0136085 A1
(43) Pub. Date: May 19, 2016

(54) WATER-BASED LIQUID COSMETIC COMPOSITIONS

(71) Applicant: L'OREAL, Paris (FR)

(72) Inventors: Christine Marie CRANE, Watchung, NJ (US); Angeles FONOLLA-MORENO, Scotch Plains, NJ (US)

(21) Appl. No.: 14/540,387

(22) Filed: Nov. 13, 2014

(51) Int. Cl. A61K 8/90; A61K 8/87; A45D 34/04; A61Q 1/10

(52) U.S. Cl. CPC A61K 8/90; A61Q 1/10; A61K 8/87; A45D 34/04; A61K 2800/872; A61K 2800/43; A61K 2800/48; A61K 2800/54; A61K 2800/5922

(57) ABSTRACT

Disclosed are water-based wax free liquid cosmetic compositions comprising a synthetic thickening polymer, a natural thickening polymer, at least one nonionic surfactant, film forming polymers and polyurethane polymer. The ratio of the synthetic thickening polymer to the natural thickening polymer is ratio is less than or equal to 4:1.

### TECHNICAL FIELD

The present invention relates to water-based, wax free liquid cosmetic compositions having a low viscosity and reduced pigment sedimentation.

### BACKGROUND OF THE INVENTION

Liquid cosmetic compositions are desirable as they are easy to apply. Water-based cosmetic compositions, in particular face and eyeliner compositions, are desirable as water based cosmetics can be easier to remove, are less likely to clog pores, may afford a less shiny and more natural look and feel, and are less expensive to process when compared to oil or solvent-based compositions. Make-up compositions for skin, in certain embodiments eyeliners, containing an aqueous medium, film forming polymers and pigments are known, for example, U.S. Pat. No. 4,423,031 and U.S. Pat. No. 6,641,823.

Currently commercially available water based eyeliners utilize latex film forming polymers, pigments, thickeners and/or waxes. However, those compositions are characterized by pigment sedimentation over time, creaming, foaming, gel formation, syneresis and phase separation, all of which are undesired physical features of the finished products. In order to obtain homogenous skin coverage those products require pre-mixing before application on the skin. In addition, they might have the appearance of not being stable.

There remains need for a water-based, wax free liquid cosmetic composition having a low viscosity that does not require pre-mixing before application and has the appearance of being stable over time.

The current invention provides water-based, wax free liquid cosmetic compositions having low viscosities, characterized by reduction or total elimination of drawbacks such as: pigment sedimentation, creaming, syneresis, foaming and phase separation. These compositions do not require pre-mixing before application on the skin or even during production. Due to their stability the compositions of the invention can be packed in clear, translucent or transparent containers.

### BRIEF SUMMARY OF THE INVENTION

The present invention relates to water-based, wax free liquid cosmetic compositions comprising at least one synthetic thickening polymer, at least one natural thickening polymer, at least one nonionic surfactant, at least one film forming polymer, at least one polyurethane polymer, water and pigment, said compositions preferably having a viscosity less than about 7 Pa·s.

In an embodiment, the invention relates to water-based, wax free liquid cosmetic compositions comprising at least one synthetic thickening polymer, at least one natural thickening polymer, at least one nonionic surfactant, at least one film forming polymer, at least one polyurethane film forming polymer, water and pigment, said compositions preferably having a viscosity less than about 7 Pa·s.

In an embodiment the ratio of the at least one synthetic thickening polymer to the at least one natural thickening polymer is less than or equal to 4:1.

In another embodiment the ratio of the at least one film forming polymer to the at least one polyurethane polymer is greater than 1:1.

In another embodiment the ratio of the at least one polyurethane polymer to the at least one natural thickening polymer is greater than 10:1.

In another embodiment the composition includes at least one first carbohydrate based ester nonionic surfactant and at least one second block-copolymer nonionic surfactant. In this embodiment, the ratio of the first carbohydrate based ester nonionic surfactant to the second block-copolymer nonionic is less than or equal to 1:2.

Another embodiment of the invention relates to a method of making up a keratinous substance, in particular the eyes and applying to the eye lids the above-described composition.

Another embodiment of the invention relates to a method of reducing or minimizing sedimentation of pigment, creaming, foaming, syneresis and/or phase separation (improving stability or pigment suspension capability).

Another embodiment of the invention relates to a method of making the water-based, wax free liquid cosmetic compositions comprising at least one synthetic thickening polymer, at least one natural thickening polymer, at least one nonionic surfactant, at least one film forming polymer, at least one polyurethane film forming polymer, water and pigment, said compositions preferably having a viscosity less than about 7 Pa·s.

The composition optionally may include other components appropriate for its intended use such as emollients, preservatives, neutralizers, vitamins, fillers, pigments and the like.

The inventive composition can be in the form of dispersion, W/O emulsion or O/W emulsion.

### DETAILED DESCRIPTION OF THE INVENTION

In the following description of the invention and the claims appended hereto, it is to be understood that the terms used have their ordinary and accustomed meanings in the art, unless otherwise specified. All percentages, ranges and ratios of components are by relative weight of actives to the total weight of the final composition.

“About” as used herein means within 10% of the indicated number (e.g. “about 10%” means 9%-11% and “about 2%” means 1.8%-2.2%).

As used herein, all ranges provided are meant to include every specific range within, and combination of sub ranges between, the given ranges. Thus, a range from 1-5, includes specifically 1, 2, 3, 4 and 5, as well as sub ranges such as and 2-5, 3-5, 2-3, 2-4, 1-4, etc.

“Centrifugation” means a process used to separate immiscible liquids or dense solids. During this process, centrifugal force (outward force) causes sedimentation of heterogeneous mixtures.

“Easy removal” means the composition may be substantially removed with a non-harsh remover, such as water and/or with a water-based cleansing solution, and without excessive rubbing.

“Film forming polymer” or “film former” as used herein means a polymer or resin that leaves a film on the substrate to which it is applied, for example, after a solvent accompanying the film former has evaporated, absorbed into and/or dissipated on the substrate.

“Liquid” means a composition that is capable of flowing its own weight, at room temperature (25° C.) and at atmospheric pressure (760 mmHg), as opposed to “solid” compositions.

“Pigment suspension capability (PSC)” or “stability” of a composition mean ability of preventing composition from pigment sedimentation or agglomeration, phase separation, foaming, creaming and gel formation.

“Surfactant” is a term of art that is well known to those skilled in the art. See, e.g. http://pharmlabs.unc.edu/labs/emulsions/agents.htm. It is a compound that has a hydrophilic part and a lipophilic part (“amphiphilic”) and facilitates the dispersion of two mutually insoluble phases and also lower surface tension.

“Thickening polymers” or “thickeners” mean substances increasing viscosity of liquids.

“Viscosity” means a measure of a fluid's resistance to flow. It describes the internal friction of a moving fluid. A fluid with “high viscosity” resists motion because its molecular makeup gives it a lot of internal friction. A fluid with “low viscosity” flows easily because its molecular makeup results in very little friction when it is in motion.

“Water based” means that the composition contains water at minimum of 5% or more.

“Wax free” means that the level of waxes in a composition is from 0 to about 0.05%, preferably 0.

In an embodiment, the invention relates to a water-based, wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer;
(d) at least one polyurethane polymer;
(e) water; and
(f) pigment;
wherein the composition has a viscosity less than about 7 Pa·s.

In another embodiment, the invention relates to a water-based, wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer;
(d) at least one polyurethane polymer;
(e) water; and
(f) pigment;
wherein the composition has a viscosity of less than about 7 Pa·s and the ratio of the at least one synthetic thickening polymer (a)(I) to the at least one natural thickening polymer (a)(II) is less than or equal to 4:1; the ratios being based on the total weight of the composition.

Preferably in another embodiment, the invention relates to a water-based, wax free liquid cosmetic composition comprising:
(a) a viscosity increasing system comprising thickening polymers selected from: (I) at least one synthetic thickening polymer, (II) at least one natural thickening polymer, and mixtures thereof;
(b) at least one nonionic surfactant;
(c) at least one film forming polymer;
(d) at least one polyurethane polymer;
(e) water; and
(f) pigment;
wherein the composition has a viscosity of less than about 7 Pa·s and the ratio of the at least one synthetic thickening polymer (a)(I) to the at least one natural thickening polymer (a)(II) is more than 1:1; the ratios being based on the total weight of the composition.

In another embodiment, the invention relates to a water-based, wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer;
(d) at least one polyurethane polymer;
(e) water; and
(f) pigment;
wherein the composition has a viscosity of less than about 7 Pa·s and the ratio of the at least one film forming polymer (c) to the at least one polyurethane polymer (d) is greater than or equals 1:1; all ratios being based on the total weight of the composition.

In another preferred embodiment, the invention relates to a water-based wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer selected from: acrylic polymers, silicone polymers, natural polymers and mixtures thereof;
(d) at least one polyurethane polymer;
(e) water; and
(f) pigment;
wherein the composition has a viscosity of less than about 7 Pa·s and the ratio of the at least one film forming polymer (c) to the at least one polyurethane polymer (d) is greater than 1:1; all ratios being based on the total weight of the composition.

In another embodiment, the invention relates to a water-based, wax free liquid cosmetic composition comprising:
(a) a viscosity increasing system comprising (I) at least one synthetic thickening polymer; and (II) at least one natural thickening polymer;
(b) at least one nonionic surfactant selected from: carbohydrate based ester nonionic surfactants, block-copolymer nonionic surfactants and mixtures thereof;
(c) at least one film forming polymer;
(d) at least one polyurethane polymer;
(e) water; and
(f) pigment;
wherein the composition has a viscosity of less than about 7 Pa·s and the ratio of the at least one carbohydrate based ester nonionic surfactant to the at least one block-copolymer nonionic surfactant is greater than or equal to 1:2; all ratios being based on the total weight on the composition.

In another embodiment, the invention relates to a water-based, wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer;
(d) at least one polyurethane polymer;
(e) water; and
(f) pigment;
wherein the composition has a viscosity of less than about 7 Pa·s and the ratio of the at least one polyurethane polymer(d) to the at least one natural thickening polymer (a)(II) is greater than or equal to 10:1; all ratios being based on the total weight of the composition.

In another embodiment, the invention relates to a water-based, wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer;
(d) at least one polyurethane polymer;
(e) water; and
(f) pigment;
wherein the composition has a viscosity of less than about 7 Pa·s and the ratio of the at least one polyurethane polymer(d) to the at least one synthetic thickening polymer (a)(I) is greater than or equal to 3:1; all ratios being based on the total weight of the composition.

In another embodiment, the invention relates to a water-based, wax free liquid cosmetic composition comprising:
(a) a viscosity increasing system comprising (I) from about 0.01% to about 1% by weight of at least one synthetic thickening polymer; and (II) from about 0.01% to about 0.5% by weight of at least one natural thickening polymer;
(b) at least one nonionic surfactant present in an amount from about 0.1% to about 5.0% by weight;
(c) at least one film forming polymer present in an amount from about 0.01% to about 20.0% by weight;
(d) at least one polyurethane polymer present in an amount from about 1% to about 15.0% by weight
(e) water present in an amount from about 5% to about 60.0% by weight; and
(f) a pigment present in an amount from about 5% to about 30.0% by weight;
wherein the composition has a viscosity of less than about 7 Pa·s, all percent ranges being based on the total weight of the composition.

In the immediately preceding embodiments, the viscosity tends to be from about 0.1 Pa·s to about 10 Pa·s, preferably from about 0.5 Pa·s to about 7 Pa·s, all ranges and sub ranges being measured at 200 rmp, using spindle number 3 or 4 (Method D described below).

In another embodiment, the invention relates to a method of reducing or minimizing undesired properties of the water-based, wax free liquid eyeliner selected from: pigment sedimentation, creaming, syneresis, foaming or phase separation. Reduction of said undesired characteristics is possible by incorporating in said eyeliner at least one synthetic thickening polymer, at least one natural thickening polymer, at least one nonionic surfactant, at least one film forming polymer and at least one polyurethane polymer. The inventive compositions are characterized by low viscosity, low gloss and their application on eyelids is easy and uniform.

In another embodiment, the invention relates to a method of making up a keratinous substance, in particular the eyes, with the above described cosmetic composition.

### Viscosity Increasing System/Thickening Polymers (a)

The thickening polymers useful in the practice of embodiments of the disclosure include those conventionally used in cosmetics. Representative thickening polymers include synthetic and natural viscosity increasing polymers.

The viscosity increasing system (a) is present in the composition of the invention in an amount of from about 0.02% to about 1.5% by weight, typically from about 0.05% to about 1% by weight, more typically from about 0.2% to about 0.9 by weight, including all ranges and sub ranges there between, all weights being based on the total weight of the composition.

Synthetic Thickening Polymer (a)(I)

These typically high molecular weight polymers increase viscosity when dissolved in the continuous phase by occupying a large volume and immobilizing the continuous phase in the polymer network. See, e.g., Paint & Coating Testing Manual, 14^{th} Edition (JV Koleske Ed., 1995), pp. 268-288; and WO2011/076792. Non-limiting examples of hydrophilic thickeners include modified or unmodified carboxyvinyl polymers, such as the products sold under the name CARBOPOL (CTFA name: carbomer) by Goodrich, homopolymers or copolymers of acrylic or methacrylic acids or the salts thereof and the esters thereof, polyacrylates and polymethacrylates such as the products sold under the names LUBRAJEL and NORGEL by Guardian, or under the name HISPAJEL by Hispano Chimica, and polyacrylic acids of SYNTHALEN K type, polyacrylamides, copolymers of acrylic acid and of acrylamide sold in the form of the sodium salt thereof, such as under the names RETEN® by Hercules, the sodium polymethacrylate such as sold under the name DARVAN 7® by Vanderbilt, and the sodium salts of polyhydroxycarboxylic acids, optionally crosslinked and/or neutralized 2-acrylamido-2-methylpropanesulphonic acid polymers and copolymers, for instance poly(2-acrylamido-2-methylpropanesulphonic acid) such as sold by Clariant under the name HOSTACERIN AMPS (CTFA name: ammonium polyacryldimethyltauramide), polyacrylic acid (available commercially as Carbomers) and acrylates copolymers such as sodium polyacrylate and polyacryloyldimehtyl taurate, and mixtures of these.

Particularly useful hydrophilic synthetic thickening polymers are crosslinked anionic copolymers of acrylamide and of AMPS, e.g. in the form of a water-in-oil emulsion, such as those sold under the name SEPIGEL™ 305 (CTFA name: Polyacrylamide/C13-14 lsoparaffin/Laureth-7, Aristoflex SNC (CTFA name: ammonium acryloyldimethyltaurate/steareth-8 methacrylate copolymer) and under the name SIMULGEL™ 600 (CTFA name: Acrylamide/Sodium acryloyldimethyltaurate copolymer/Isohexadecane/Polysorbate 80) by SEPPIC.

The other useful synthetic thickening polymers include sodium acryloyldimethyltaurate/VP crosspolymer, acrylates copolymer, sodium polyacrylate, ammonium acryloyl dimethyltaurate/carboxyethyl acrylate crosspolymer, ammonium polyacryloyldimethyl taurate, ammonium acryloyldimethyltaurate/vinyl formamide copolymer, ammonium polyacryloyldimethyl taurate/ammonium polyacryldimethyltauramide and ammonium acryloyldimethyltaurate/VP copolymer, and mixtures thereof.

In a preferred embodiment, the synthetic thickening polymer is SIMULGEL™ 600 (CTFA name: Acrylamide/Sodium acryloyldimethyltaurate copolymer/Isohexadecane/Polysorbate 80) by SEPPIC and/or Aristoflex SNC (CTFA name: ammonium acryloyldimethyltaurate/steareth-8 methacrylate copolymer).

In an embodiment, the hydrophilic synthetic thickening polymer (a)(I) is present in the composition of the invention in an amount of from about 0.01% to about 1.0% by weight, particularly from about 0.1% to about 0.8% by weight, more particularly from about 0.2% to about 0.7% by weight, including all ranges and sub ranges there between, all weights being based on the total weight of the composition.

Hydrophilic thickeners applicable in this invention include poly(N-vinyl)lactam polymers and its derivatives. Such polymers are known and have been disclosed in U.S. Pat. No. 4,642,267, the entire content of which is hereby incorporated by reference.

Particularly useful hydrophilic poly(N-vinyl)lactam thickener is polyvinylpyrrolidone/PVP (sold under PVP K 30L available from ISP and LUVISKOL K 30 available from BASF), VINYLPYRROLIDONE/VINYLACETATE COPOLYMER (sold under PVP/VA S 630 L, available from ISP), VINYL CAPROLACTAM/VP/DIMETHYLAMINOETHYL METHACRYLATE COPOLYMER (sold under ADVANTAGE HC 37, available from ISP.

Synthetic thickening polymers also include non-crosslinked or crosslinked polymers that are capable of non-specific hydrophobic associations due to polymers' hydrophobic modification. These hydrophobic associations are chiefly responsible for the increase in viscosity observed with these thickeners. See Paint & Coating Testing Manual, 14^{th} Edition (JV Koleske Ed., 1995), pp. 268-288. In the current invention, the synthetic associative thickening polymers used unexpectedly modified the rheology profile of the resulting composition to afford high pseudoplasticity (that is increased flowability of the compositions at high shear and enhanced product pick-up). Further, incorporation of these polymers resulted in compositions having a larger slope in the corresponding viscosity (Pa's) versus shear rate (1/s) graph as determined on a TA Instrument G2 Rheometer with a 20 mm 2° cone in continuous or steady state mode. Examples of such polymers are provided in U.S. Pat. No. 7,220,408, which is herein incorporated by reference. See, also, Paint & Coating Testing Manual, 14^{th} Edition (JV Koleske Ed., 1995), pp. 268-288

The synthetic associative polymers in accordance with various exemplary embodiments may be anionic, cationic, nonionic or amphoteric. By way of example, synthetic associative polymers which may be chosen include those comprising at least one hydrophilic unit and at least one fatty-chain allyl ether unit, such as those in which the hydrophilic unit is constituted of an ethylenic unsaturated anionic monomer, such as a vinylcarboxylic acid or an acrylic acid, a methacrylic acid, and mixtures thereof, and in which the fatty-chain allyl ether unit corresponds to the monomer of formula (I) below:

CH₂═C(R′)CH₂OBₙR (I)
in which R′ is chosen from H or CH₃, B is chosen from an ethyleneoxy radical, n is zero or is chosen from an integer ranging from 1 to 100, and R is chosen from a hydrocarbon-based radical chosen from alkyl, arylalkyl, aryl, alkylaryl and cycloalkyl radicals containing from 8 to 30 carbon atoms, such as from 10 to 24 carbon atoms, or from 12 to 18 carbon atoms. Exemplary and non-limiting polymers of this type are described and prepared, according to an emulsion polymerization process, in patent EP 0 216 479.

Non-limiting examples of synthetic associative anionic polymers that may also be chosen include anionic polymers comprising at least one hydrophilic unit of olefinic unsaturated carboxylic acid type, and at least one hydrophobic unit exclusively of (C₁₀-C₃₀)alkyl ester of unsaturated carboxylic acid type. Examples that may be mentioned include, but are not limited to, the anionic polymers described and prepared according to patents U.S. Pat. Nos. 3,915,921 and 4,509,949, which are herein incorporated by reference.

Cationic associative polymers that may be chosen include, but are not limited to, polyacrylates containing amine side groups.

Exemplary non-ionic associative polymers include copolymers of vinylpyrrolidone and of fatty-chain hydrophobic monomers, for instance Antaron® or Ganex® V216 (vinylpyrrolidone/hexadecene copolymers); Antaron® or Ganex® V220 (vinylpyrrolidone/eicosene copolymers), sold by the company I.S.P., copolymers of C₁-C₆ alkyl methacrylates or acrylates and of amphiphilic monomers comprising at least one fatty chain, and copolymers of hydrophilic methacrylates or acrylates and of hydrophobic monomers comprising at least one fatty chain, for instance the polyethylene glycol methacrylate/lauryl methacrylate copolymer; polymers with an aminoplast ether skeleton containing at least one fatty chain, such as the Pure Thix® nonionic associative water phase thickeners sold by the company Southern Clay Products, Inc.

Associative polyurethanes may also be chosen in various exemplary and non-limiting embodiments. These are nonionic block copolymers comprising in the chain both hydrophilic blocks usually of polyoxyethylene nature, and hydrophobic blocks that may be aliphatic sequences alone and/or cycloaliphatic and/or aromatic sequences. Associative polyurethanes comprise at least two hydrocarbon-based lipophilic chains containing from C₆ to C₃₀ carbon atoms, separated by a hydrophilic block, the hydrocarbon-based chains optionally being pendent chains or chains at the end of a hydrophilic block. For example, it is possible for one or more pendent chains to be provided. In addition, the polymer may comprise a hydrocarbon-based chain at one or both ends of a hydrophilic block. The associative polyurethanes may be arranged in triblock or multiblock form. The hydrophobic blocks may thus be at the each end of the chain (for example, triblock copolymer with a hydrophilic central block) or distributed both at the ends and within the chain (for example, multiblock copolymer). These polymers may also be graft polymers or starburst polymers. For example, the associative polyurethanes may be triblock copolymers in which the hydrophilic block is a polyoxyethylene chain containing from 50 to 1000 oxyethylene groups.

By way of non-limiting example, associative polymers of the polyurethane polyether type that may be used include the polymer C₁₆-OE₁₂₀-C₁₆ from Servo Delden (under the name SER AD FX1100), which is a molecule containing a urethane function and having a weight-average molecular weight of 1300), OE being an oxyethylene unit, Nuvis® FX 1100 (European and US INCI name “Steareth-100/PEG-136/HMDI Copolymer” sold by the company Elementis Specialties), and also Acrysol RM 184® (sold by the company Rohm and Haas). Further exemplary associative polymers that may be chosen include RHEOLATE® 205 containing a urea function, sold by Rheox, or RHEOLATE® 208 or 204, or RHEOLATE® FX1100 from Elementis. The product DW 1206B from Rohm & Haas containing a C₂₀ alkyl chain with a urethane bond, sold at a solids content of 20% in water, may also be used.

In further exemplary embodiments, solutions or dispersions of the above-mentioned polymers, especially in water or in water-alcohol medium, may be chosen. Examples of such polymers include SER AD FX1010, SER AD FX1035 and SER AD 1070 from Servo Delden, and RHEOLATE® 255, RHEOLATE® 278 and RHEOLATE® 244 sold by Rheox. Further examples include the products ACULYN™ 46, DW 1206F and DW 1206J, and also ACRYSOL RM 184 or ACRYSOL 44 from Rohm & Haas, and BORCHIGEL LW 44 from Borchers.

Additional associative thickening polymers include polyacrylic acid/alkyl acrylate copolymers of PEMULEN type; PEG-150/stearyl alcohol/SMDI copolymer such as that sold under the name ACULYN™ 46 by Rohm & Haas; steareth-100/PEG-136/HDI copolymer such as sold under the name RHEOLATE® FX 1100 by Elementis).

Mixtures of the above associative polymers are also contemplated herein as useful in the invention.

As used herein, the term “copolymers” is intended to mean both copolymers obtained from two types of monomers and those obtained from more than two types of monomers, such as, for example, terpolymers obtained from three types of monomers. The chemical structure of the copolymers comprises at least one hydrophilic unit and at least one hydrophobic unit. The expression “hydrophylic unit” or “hydrophobic unit” is understood to mean a radical possessing a saturated or unsaturated and linear or branched hydrocarbon-based chain which comprises at least 8 carbon atoms, for example from 10 to 30 carbon atoms, as a further example from 12 to 30 carbon atoms, and as yet a further example from 18 to 30 carbon atoms.

In certain exemplary and non-limiting embodiments, the associative thickening copolymers are chosen from the copolymers resulting from the polymerization of:

(1) at least one monomer of formula (II):

CH2=CH(R1)COOH (II)
wherein R₁ is chosen from H or CH₃ or C₂H₅, providing acrylic acid, methacrylic acid, or ethacrylic acid monomers, and
   (2) at least one monomer of (C₁₀-C₃₀)alkyl ester of unsaturated carboxylic acid type corresponding to the monomer of formula (III):

CH2=CH(R2)COOR3 (III)
wherein R₂ is chosen from H or CH₃ or C₂H₅, providing acrylate, methacrylate or ethacrylate units, R₃ denoting a C₁₀-C₃₀ alkyl radical, such as a C₁₂-C₂₂ alkyl radical.

Non-limiting examples of (C₁₀-C₃₀)alkyl esters of unsaturated carboxylic acids are for example chosen from lauryl acrylate, stearyl acrylate, decyl acrylate, isodecyl acrylate, dodecyl acrylate and the corresponding methacrylates, such as lauryl methacrylate, stearyl methacrylate, decyl methacrylate, isodecyl methacrylate and dodecyl methacrylate, and mixtures thereof.

Additionally, crosslinked thickening polymers may be chosen according to further exemplary embodiments. For example, such polymers may be chosen from polymers resulting from the polymerization of a mixture of monomers comprising:
acrylic acid,
an ester of formula (III) described above, in which R₂ is chosen from H or CH₃) R₃ denoting an alkyl radical having from 12 to 22 carbon atoms, and
a crosslinking agent, which is a well-known copolymerizable polyethylenic unsaturated monomer, such as diallyl phthalate, allyl (meth)acrylate, divinylbenzene, (poly)ethylene glycol dimethacrylate and methylenebisacrylamide.

By way of example, crosslinked thickening polymers comprising about 60% to about 95% by weight of acrylic acid (hydrophilic unit), about 4% to about 40% by weight of C₁₀-C₃₀ alkyl acrylate (hydrophobic unit), and about 0% to about 6% by weight of crosslinking polymerizable monomer.

In yet further embodiments, the crosslinked thickening polymers may comprise about 96% to about 98% by weight of acrylic acid (hydrophilic unit), about 1% to about 4% by weight of C₁₀-C₃₀ alkyl acrylate (hydrophobic unit), and about 0.1% to 0.6% by weight of crosslinking polymerizable monomer, such as those described above. Examples of such polymers include acrylate/C₁₀-C₃₀ alkyl acrylate copolymers (INCI name: Acrylates/C10-30 Alkyl Acrylate Crosspolymer), such as the products sold by Lubrizol under the trade names PEMULEN™ TR1, PEMULEN™ TR2, CARBOPOL® 1382 and CARBOPOL® EDT 2020 may be chosen.

In additional embodiments, the at least one synthetic associative thickening polymer may be chosen from nonionic homopolymers or copolymers containing ethylenically unsaturated monomers of the ester and/or amide type. For example, the products sold under the names CYANAMER P250 by the company CYTEC (polyacrylamide), methyl methacrylate/ethylene glycol dimethacrylate copolymers (such as PMMA MBX-8C by the company US COSMETICS), butyl methacrylate/methyl methacrylate copolymers (such as ACRYLOID B66 by the company RHOM HMS), and polymethyl methacrylates (BPA 500 by the company KOBO) may be chosen.

Further, non-limiting examples of synthetic associative thickening polymers include polyacrylamide(and)C13-14 isoparaffin(and)laureth-7 (such as Sepigel™ 305 from Seppic), acrylates/C10-30 alkyl acrylate crosspolymer (such as Carbopol® Ultrez 20 polymer from Lubrizol), acrylates/C10-30 alkyl acrylate crosspolymer (such as Permulen™ TR-1 from Lubrizol), and polyacrylate crosspolymer-6 (such as Sepimax Zen from Seppic).

In a preferred embodiment, the associative thickening polymers include cross- or co-polymers of polyacryloyl/taurate or polyacryloyl/dimethyltaurate. Non-limiting examples of such polymers include ammonium acryloyldimethyltaurate/steareth-25 methacrylate crosspolymer, ammonium acryloyldimethyltaurate/steareth-8 methacrylate copolymer, ammonium acryloyldimethyltaurate/beheneth-25 methacrylate crossopolymer, and ammonium acryloyldimethyltaurate/laureth-7 methacrylate copolymer, and mixtures thereof.

Additional non-limiting examples of useful associative thickening polymers include acrylates/vinyl neodecanoate crosspolymer, acrylates/steareth-20 methacrylate crosspolymer, and peg-150/stearyl alcohol/SMDI copolymer, and mixtures thereof.

Natural Thickening Polymers (a)(II)

Natural thickening polymers may be selected from, for example vegetable gums, liposoluble/lipodispersible polymers, salts, and mixtures thereof. Preferably the viscosity increasing agents are not waxes

Representative natural viscosity increasing polymers (agents) that may be used in the practice of embodiments according to the disclosure may be chosen from nonionic, anionic, cationic, and amphoteric polymers, polysaccharides, polyamino compounds, amphiphilic polymers, and other viscosity modifiers such as cellulose-based thickeners (e.g. microcrystalline, hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, cross-linked homopolymers of acrylic acid or of acrylamidopropane-sulfonic acid, associative polymers, non-associative thickening polymers, water-soluble thickening polymers, and mixtures of these.

In a preferred embodiment, the natural thickening polymers are CELLOSIZE QP 4400 H (INCI name: hydroxycellulose) by AMERCHOL, RHODICARE XC (INCI NAME: xanthan gum) by RODIA, KELTROL CG (INCI NAME: xanthan gum) by CP KELCO or their mixtures.

In an embodiment of the invention the natural thickening polymer (a)(II) is present in an amount from about 0.01% to about 0.5%, more particularly from about 0.05% to about 0.4%, preferably from about 0.1% to about 0.25% by weight, based on the weight percent of the thickening polymer in the final composition.

In an embodiment of the invention, the ratio of the at least one synthetic thickening polymer (a)(I) to the at least one natural thickening polymer (a)(II) is lower than 7:1, more particularly from about 5:1 to about 4:1, even more particularly from about 3.5:1 to about 2:1 by weight, based on the weight percent of each thickening polymer in the final composition.

### Surfactants (b)

The composition according to the invention comprises at least one nonionic surfactant.

The composition in accordance with the invention comprises at least one nonionic surfactant present in an amount ranging from about 0.1% to about 5% by weight, preferably from about 0.2% to about 3% and more particularly from 1% to 2.5% by weight, relative to the weight of the composition.

According to the invention, use is made of nonionic surfactants appropriately chosen in order to obtain a wax free liquid composition with decreased or eliminated pigment sedimentation (increased stability). Use may in particular be made of surfactants having, at 25 degrees centigrade, an HLB balance (hydrophilic-lipophilic balance), within the Griffin meaning, of greater than or equal to 8, more particularly greater than or equal to 10 HLB, preferably greater than or equal to 13 HLB used alone or as a mixture.

The HLB value according to Griffin is defined in J. Soc. Cosm. Chem., 1954 (volume 5), pages 249-256.

Mention may in particular be made of:
monosaccharide esters and ethers, such as the mixture of cetylstearyl glucoside and of cetyl and stearyl alcohols, for example Montanov 68 from Seppic;
oxyethylenated and/or oxypropylenated ethers (which can comprise from 1 to 150 oxyethylene and/or oxypropylene groups) of glycerol; oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of cetearyl alcohol comprising 30 oxyethylene groups (CTFA name “Ceteareth-30”), the oxyethylenated ether of stearyl alcohol comprising 20 oxyethylene groups (CTFA name “Steareth-20”) and the oxyethylenated ether of the mixture of C₁₂-C₁₅ fatty alcohols comprising 7 oxyethylene groups (CTFA name “012-15 Pareth-7”), sold under the name Neodol 25-7® by Shell Chemicals;
esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniquema;
esters of fatty acid (in particular of C₈-C₂₄ and preferably C₁₆-C₂₂ acid) and of oxyethylenated and/or oxypropylenated glycerol ethers (which can comprise from 1 to 150 oxyethylene and/or oxypropylene groups), such as PEG-200 glyceryl monostearate, sold under the name Simulsol 220™® by Seppic; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat 0® sold by Goldschmidt, polyethoxylated glyceryl cocoate comprising 30 ethylene oxide groups, such as the product Varionic LI 13® sold by Sherex, polyethoxylated glyceryl isostearate comprising 30 ethylene oxide groups, such as the product Tagat L® sold by Goldschmidt, and polyethoxylated glyceryl laurate comprising 30 ethylene oxide groups, such as the product Tagat 1® from Goldschmidt; esters of fatty acid (in particular of C₈-C₂₄ and preferably C₁₆-C₂₂ acid) and of oxyethylenated and/or oxypropylenated sorbitol ethers (which can comprise from 1 to 150 oxyethylene and/or oxypropylene groups), commonly known as “carbohydrate based esters (CBEs)”. The examples of said carbohydrate based esters, but not limited are such as polysorbate 20, sold under the name Tween20® by Croda, polysorbate 21, sold under the name Tween21® by Croda or polysorbate 60, sold under the name Tween60® by Croda; dimethicone copolyol, such as that sold under the name Q2-5220® by Dow Corning; dimethicone copolyol benzoate (Finsolv SLB 101® and 201® from Fintex). Examples of such surfactants are provided in US 2012/042894, which is herein incorporated by reference. Preferably useful in this inventive composition is polysorbate 21 (Tween 21 from Croda), which is present from about 0.2% to about 0.7% by weight, relative to the weight of the composition.

In the embodiment the non-ionic surfactants are selected from block-copolymer surfactants, also known as polycondensate surfactants of ethylene oxide and of propylene oxide and more particularly a copolymer consisting of polyethylene glycol and polypropylene glycol blocks, such as, for example, polyethylene glycol/polypropylene glycol/polyethylene glycol triblock block-copolymers (polycondensates). These triblock polycondensates have, for example, the following chemical structure:

H—(O—CH₂—CH₂)ₐ—(O—CH(CH₃)—CH₂)_{b}—(O—CH₂—CH₂)ₐ—OH
in which formula a ranges from 2 to 150 and b ranges from 1 to 100; preferably, a ranges from 10 to 130 and b ranges from 20 to 80.

The block-copolymer (polycondensate) surfactant of ethylene oxide and of propylene oxide preferably has a weight-average molecular weight ranging from 1000 to 20000, better still ranging from 1500 to 19000, in particular ranging from 2000 to 18000 and even better still ranging from 4000 to 17000.

Mention may be made, as block-copolymer (polycondensate) surfactant of ethylene oxide and of propylene oxide which can be used according to the invention, of the polyethylene glycol/polypropylene glycol/polyethylene glycol triblock polycondensates sold under the “Synperonic” names, such as “Synperonic® PE/F32” (INCI name: Poloxamer 108), “Synperonic® PE/F108” (INCI name: Poloxamer 338), “Synperonic® PE/L44” (INCI name: Poloxamer 124), “Synperonic® PE/L42” (INCI name: Poloxamer 122), “Synperonic® PE/F127” (INCI name: Poloxamer 407), “Synperonic® PE/F88” (INCI name: Poloxamer 238) or “Synperonic® PE/L64” (INCI name: Poloxamer 184), by Croda or also “Lutrol® F68” (INCI name: Poloxamer 188), sold by BASF

The block-copolymer (polycondensate) surfactant of ethylene oxide and of propylene oxide can be present in the composition according to the invention in a content ranging for example from 0.01 percent to 5 percent by weight, with respect to the total weight of the composition, preferably ranging from 0.05 percent to 3 percent by weight and preferentially ranging from 0.05 percent to 1 percent by weight.

Specifically useful in this inventive composition is block-copolymer known as Poloxamer 338, which is present from about 0.1% to about 2%, preferably from about 0.25% to about 1.2% by weight, relative to the weight of the composition. Examples of polycondensate surfactants beneficial in this invention are provided in US 2009/232756 and http://www.pharma-ingredients.basf.com/Statements/Technical%20Informations/EN/Pharma%20Solutions/03_111136e_Kolliphor%20P%20Grades.pdf which are herein incorporated by reference.

### Film-Forming Polymer (c)

As used herein, the terms “film-forming polymer,” “film former,” “film-forming agent” and variations thereof mean a polymer capable of, by itself or in the presence of an auxiliary film-forming polymer, forming a continuous film that adheres to a support and especially to keratin materials, for instance the eyes.

In the above embodiment the solid content of film forming polymer is typically present in an amount from about 0.01% to about 20% by weight, preferably from about 5% to about 19% by weight, more particularly from about 9% to about 18% by weight, relative to the weight of the composition.

The film-forming polymers that may be used in the cosmetic compositions disclosed herein include, for example, acrylate copolymers, styrene/acrylate copolymers, acrylaminde/acrylate copolymers, polyurethanes, silicone resins and block silicone copolymer particles, and other resins, and derivatives thereof and mixtures thereof.

In accordance with various exemplary embodiments, acrylate copolymers may be chosen from copolymers comprising two or more monomers chosen from acrylic acid, methacrylic acid, and their simple esters, for example, lower alkyl esters such as methyl, ethyl, and ethylhexyl esters.

By way of non-limiting example only, acrylate copolymers may be chosen from styrene acrylates/copolymers, ammonium acrylates copolymers, ethyl acrylates copolymers, acrylates/ethylhexylacrylate copolymers, acrylates/octylacrylates copolymers, alkyl (meth)acrylates copolymers, acrylates/C₁₂-C₂₂ alkylmethacrylate copolymers, ethylacrylate/methacrylic acid copolymer, and t-butyl acrylate/ethyl acrylate/methacrylic acid copolymer. Exemplary commercial acrylate copolymers include, but are not limited to, ALLIANZ™ OPT sold by Ashland Specialty Ingredients; COVACRYL A15 and COVACRYL E14 sold by Sensient Cosmetic Technologies LCW; DAITOSOL 4000 SJT, DAITOSOL 5000 AD, DAITOSOL 5000 SJ, KOBOGUARD® 50A, and KOBOGUARD® 50N sold by Kobo Products, Inc.; DERMACRYL® AQF, YODOSOL 32A707, YODOSOL GH15, YODOSOL GH32, YODOSOL GH33, YODOSOL GH34, YODOSOL GH35, YODOSOL GH800, and YODOSOL GH810 sold by AkzoNobel; LUVIFLEX® SOFT, LUVIMER® 36D, and LUVIMER® 100P sold by BASF; and NEOCRYL XK-90 sold by Neoresins, Inc

The film forming polymer may also be chosen from polyacrylates such as polyacrylate-21, and polyacrylate-15, and acrylates copolymer.

In another embodiment the at least one film forming polymer can be selected from a lipophilic polymer, such as for example, poly C₁₀₋₃₀ alkylacrylates (available as Intelimer® IPA 13-1 from Air Products).

### Latex Film Formers

The film-forming polymer may also be chosen from latex film forming polymers such as polyacrylate latex and their copolymers.

Suitable examples of latex polymers for use in the present invention are ethylhexyl acrylate/hema copolymer (and) acrylates/diethylaminoethyl methacrylate/ethylhexyl acrylate copolymer (Syntran® PC 5775), styrene/acrylates/ammonium methacrylate copolymer (Syntran® 5760, Syntran® 5009, Syntran® PC5620), polyacrylate-21 (and) acrylates/di methylaminoethyl methacrylate copolymer (Syntran® PC5100, Syntran® PC5776, Eudragit E 100, Jurymer ET-410C), styrene/acrylates/ammonium methacrylate copolymer (Syntran® 5009 CG), olefin/acrylate grafted polymer (and) sodium laureth sulfate (and C12-15 SEC-pareth 15 (Syntran® EX108), acrylates copolymer (Aculyn® 33A Polymer, Avalure® Ace 210/120/315 Acrylic Copolymer, Carbopol Aqua SF-1® Polymer, Daitosol® 500 AD, Coatex® Co 633, Eliclear® 380/700/4U, Eudragit® L 100, Joncryl® 85, Luviflex® Soft), acrylates/ethylhexyl acrylate copolymer (Daitosol® 5000SJ, Daitosol® 4000SJT, MJA PS34-21, SDP-001). The Syntran® polymers are commercially available from the supplier Interpolymer Corp.

In an embodiment, the latex polymer is an acrylate latex polymer, in particular styrene/acrylate copolymers. Non-limiting examples of commercially available styrene/acrylate copolymers include, but are not limited to, DAITOSOL 5000 STY sold by Kobo Products, Inc.; JONCRYL® 77 sold by BASF; NEOCRYL BT-62 sold by Neoresins, Inc.; RHOPLEX™ P-376 and UCAR™ DL 432S sold by Dow Chemical Company; and YODOSOL GH41 and YODOSOL GH840 sold by AkzoNobel.

In further exemplary embodiments, acrylamide/acrylate copolymers may be chosen from acrylic acid/ethyl acrylate/t-butyl acrylamide copolymer, acrylates/octylacrylamide copolymer, and octylacrylamide/acrylates/methacrylates copolymer. Exemplary commercial acrylamide/acrylate copolymers include, but are not limited to AMPHOMER® LV-71 and DERMACRYL® 79 sold by AkzoNobel and ULTRAHOLD® STRONG sold by BASF.

In at least one exemplary embodiment, the latex film former may be chosen from blends comprising a combination of latex film formers, including, for example, a blend of any of the above-mentioned film formers. By way of non-limiting example, a latex film forming blend useful according to the disclosure may comprise (I) at least one random styrene acrylate copolymer or derivatives thereof, and at least one acrylate copolymer or derivatives thereof, or (2) at least two random styrene acrylate copolymers or derivatives thereof.

In an embodiment the film former is selected from styrene/acrylates/ammonium methacrylate copolymers sold by Interpolymer Corporation, in particular SYNTRAN® 5760 (styrene/acrylates/ammonium methacrylate copolymer (and) sodium laureth sulfate (and) caprylyl glycol); SYNTRAN® 5775 (acrylates/ethylhexyl acrylate/hema copolymer (and) acrylates/diethylaminoethylmethacrylate/ehtylhexyl acrylate copolymer (and) isodeceth-6 (and) caprylyl glycol (and) sodium laureth sulfate); SYNTRAN® Ex 108 (olefin/acrylate grafted polymer (and) sodium laureth sulfate (and) C12-15 SEC-pareth 15); and SYNTRAN® 108 GC (olefin/acrylic grafted emulsion).

According to at least certain exemplary embodiments of the disclosure, the at least one latex film former may be chosen from those having a glass transition temperature (Tg) ranging from about −15° C. to about 90° C., such as from about 0° C. to about 50° C.

Specifically useful in this inventive composition is styrene/acrylates copolymer such as styrene/acrylates/ammonium methacrylate copolymer (and) sodium laureth sulfate (and) caprylyl glycol (known as SYNTRAN® 5760), which is present from about 5% to about 15% by weight of solid content (active), relative to the weight of the composition.

### Silicone Resins

In accordance with other exemplary embodiments, silicone resins and copolymers may also be used as a film forming agent. In a particular embodiment, the at least one film forming polymer is a silicone copolymer.

Film-forming silicone resins and copolymers are described, for example, in U.S. Pat. No. 7,790,148 (L'Oreal), U.S. Pat. No. 7,094,842 (L'Oreal), US2011/0189,117 (L'Oreal), US2010/0297050 (L'Oreal), US 2007/0093619 and 2006/0013791, 2005/0201961, all of which are herein incorporated by reference.

Exemplary film forming silicone resins are cross-linked polyorganosiloxane polymers. The nomenclature of silicone resins is known under the name “MDTQ”, the resin being described as a function of the various monomeric siloxane units it includes, each of the letters “MDTQ” characterizing one type of unit. Examples of commercially available polymethylsilsesquioxane resins that may be mentioned are those that are sold by the supplier Wacker under the reference Resin MK such as Belsil® PMS MK, and by the supplier SHIN-ETSU under the references KR-220L.

Examples of commercially available polypropylsilsesquioxane resins useful in the invention include those sold under the reference DC0670 by the supplier Dow Corning, and Silform® Flexible Resin from the supplier Momentive.

Examples of a polyphenylsilsesquioxane resins useful in the invention include those available from Wacher.

Siloxysilicate resins that may be used include trimethylsiloxysilicate resins (TMS®) such as those sold under the reference SR1000 by the supplier Momentive Performance Materials or under the reference TMS® 803 by the supplier Wacker. Trimethylsiloxysilicate resins are also available in a solvent such as cyclomethicone, sold under the name “KF-7312J” by the supplier Shin-Etsu, or “DC® 749”, “DC® 593” by the supplier Dow Corning.

Pressure-sensitive adhesive silicone copolymers are also herein contemplated. Such copolymers are available, for example from Dow Corning under the reference BIO-PSA and described in U.S. Pat. No. 5,162,410.

Silicone copolymers derived from the reaction of a silicone resin such as those described above and of a diorganosiloxane such as that described in the document WO 2004/073626 are also contemplated.

### Block Silicone Copolymer Particles

Block silicone copolymer particles are also useful in preparing films according to the invention. These silicone compounds are described in U.S. Pat. No. 7,094,842. The silicone copolymer constituting the globules or particles in dispersion in the aqueous phase is a substantially linear block copolymer, that is to say a non-crosslinked copolymer, obtained by chain extension and not by crosslinking.

The aqueous dispersion of particles of block copolymer is a silicone-in-water emulsion (Sil/W) wherein the oily globules are constituted from a silicone of high viscosity, so that these globules seem to form as “soft particles”.

The composition may comprise dispersions of one or more types of substantially linear block silicone copolymer. These block silicone copolymers are present in the composition of the invention in concentrations, as active material, which may vary widely depending on the other ingredients of the composition and the desired aim. The concentration, as active material of block silicone copolymer preferably ranges from 0.01 to 15% by weight, even better from 0.1 to 10% by weight, and better still from 0.5 to 5% by weight, including all ranges and sub ranges therebetween, relative to the total weight of the composition.

The size of the block silicone copolymer particles vary. Preferably, in the present invention, the silicone copolymer particles generally have a number-average size of less than or equal to 2 microns, and preferably of less than or equal to 1 micron.

The aqueous dispersions of substantially linear block silicone copolymer particles used according to the invention may be chosen in particular from those described in the document EP-A-874017, whose teaching is incorporated into the present by reference. According to this document, it is possible in particular to obtain the silicone copolymers constituting these particles by chain extension reaction, in the presence of a catalyst, from at least:
(a) one polysiloxane (i) having at least one reactive group and preferably one or two reactive groups per molecule; and
(b) one organosilicone compound (ii) which reacts with the polysiloxane (i) by chain extension reaction.

In a particular embodiment, the polysiloxane (i) is chosen from a compound of formula (V):

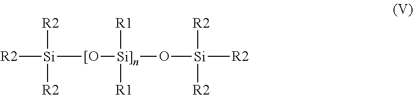

wherein R₁ and R₂, independently of each other, represent a hydrocarbon group having from 1 to 20 carbon atoms and preferably from 1 to 10 carbon atoms, such as methyl, ethyl, propyl or butyl, or an aryl group such as phenyl, or a reactive group, n is an integer greater than 1, provided that there is on average between one and two reactive groups per polymer.

As used above in defining formula (V), the expression “reactive group” is understood to mean any group capable of reacting with the organosilicone compound (ii) to form a block copolymer. As reactive groups, there may be mentioned hydrogen; aliphatically unsaturated groups and in particular vinyl, allyl or hexanyl groups; the hydroxyl group; alkoxy groups such as methoxy, ethoxy or propoxy; alkoxyalkoxy groups; the acetoxy group; amino groups, and mixtures thereof. Preferably, more than 90%, most preferably more than 98% of reactive groups are at the chain end, that is to say that the radicals R₂ generally constitute more than 90% and even 98% of the reactive groups.

Preferably, n is such that the polysiloxanes have a viscosity ranging from about 1 to 1×10⁶ mm²/sec at 25° C. n may be for example an integrer ranging from about 5 to 30, preferably from 10 to 30 and better from 15 to 25.

The polysiloxanes of formula (V) are substantially linear polymers, that is to say containing few branches, and generally less than 2 mol % of the siloxane units. Moreover, the groups R₁ and R₂ may be optionally substituted with amino groups, epoxy groups, groups containing sulphur, silicon or oxygen.

Preferably, at least 80% of the groups R₁ are alkyl groups and even better methyl groups.

Preferably, the reactive group R₂ at the chain end is an aliphatically unsaturated group and in particular a vinyl group.

As polysiloxanes (i), there may be mentioned in particular dimethylvinylsiloxypolydimethylsiloxane, a compound of formula (V) in which the radicals R₁ are methyl radicals, and, at the chain end, the radical R₂ is a vinyl radical while the other two radicals R₂ are methyl radicals.

The organosilicone compound (ii) may be chosen from the polysiloxanes of formula (V) or compounds acting as chain extension agent. If it is a compound of formula (V), the polysiloxane (i) will contain a first reactive group and the organosilicone compound (ii) will contain a second reactive group which will react with the first. If it is a chain extension agent, it may be a silane, a siloxane (disiloxane or trisiloxane) or a silazane. Preferably, the organosilicone compound (ii) is a liquid organohydrogenpolysiloxane of formula (VI):

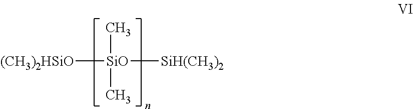

wherein “n” is an integer greater than 1 and preferably greater than 10, and for example ranging from 5 to 30, preferably from 10 to 30, and better from 15 to 25. According to a particular embodiment of the invention, “n” is equal to 20.

The block silicone copolymers used according to the invention are advantageously free from oxyalkylenated groups, especially free from oxyethylenated and/or oxypropylenated groups.

The catalyst of the reaction between the polysiloxane and the organosilicone compound may be chosen from metals and in particular from platinum, rhodium, tin, titanium, copper and lead. It is preferably platinum or rhodium.

The dispersion of silicone copolymer particles used according to the invention may in particular be obtained, for example, by mixing (a) water, (b) at least one emulsifier, (c) the polysiloxane (i), (d) the organosilicone compound (ii) and (e) a catalyst. Preferably, one of the constituents (c), (d) or (e) is added last to the mixture so that the chain extension reaction only starts in the dispersion.

As emulsifiers which may be used in the method of preparation described above for obtaining the aqueous dispersion of particles, there may be mentioned non-ionic or ionic (anionic, cationic or amphoteric) emulsifiers. They are preferably non-ionic emulsifiers which may be chosen from polyalkylene glycol ethers of a fatty alcohol, containing from 8 to 30 carbon atoms and preferably from 10 to 22 carbon atoms; polyoxyalkylenated and in particular polyoxyethylenated alkyl esters of sorbitan, where the alkyl radical contains from 8 to 30 carbon atoms and preferably from 10 to 22 carbon atoms; polyoxyalkylenated and in particular polyoxyethylenated alkyl esters, where the alkyl radical contains from 8 to 30 carbon atoms and preferably from 10 to 22 carbon atoms; polyethylene glycols; polypropylene glycols; diethylene glycols; and mixtures thereof. The quantity of emulsifier(s) is generally from 1 to 30% by weight relative to the total weight of the reaction mixture.

The emulsifier used to obtain the aqueous dispersion of particles is preferably chosen from polyethylene glycol ethers of fatty alcohols and mixtures thereof, in particular polyethylene glycol ethers of alcohols containing 12 or 13 carbon atoms or from 2 to 100 oxyethylenated units and preferably from 3 to 50 oxyethylenated units, and mixtures thereof. There may be mentioned, for example, C₁₂-C₁₃ Pareth-3, C₁₂-C₁₃ Pareth-23 and mixtures thereof.

According to a particular embodiment of the invention, the dispersion of particles of silicone copolymer is obtained from dimethylvinylsiloxypolydimethylsiloxane (or divinyldimethicone) as compound (i), and from the compound of formula (II) with preferably n=20, as compound (ii), preferably in the presence of a platinum-type catalyst, and the dispersion of particles is preferably obtained in the presence of C₁₂-C₁₃ Pareth-3 and C₁₂-C₁₃ Pareth-23 as emulsifiers.

A non-limiting example of dispersion of particles of silicone copolymer is divinyldimethicone/dimethicone copolymer (and) C12-13 pareth-23 (and) C12-13 pareth-3 (INCI name, commercially available as HMW 2220 from Dow Corning; containing about 60% by weight of active ingredient of divinyldimethicone/dimethicone copolymer, present in the inventive compositions from about 0.1% to about 1% by weight, preferably from about 0.15% to about 0.8%, most preferably from about 0.2% to about 0.7%, relative to the weight of the composition.

### Natural Polymers

In another embodiment the film forming polymer may also be selected from natural polymers (resins). For the purposes of the invention, the expression “of natural origin” is intended to denote polymeric agents that are obtained by modification of natural polymeric agents.

Among the preferred natural polymers, there may be mentioned the polymers of plant origin, the polymers derived from superficial body growths, egg proteins, latexes of natural origin and polysaccharides, and combinations thereof.

The preferred polymers of plant origin include, for example, the protein extracts of cereals, legumes and oilseeds such as extracts of maize, rye, wheat, buckwheat, sesame, spelt, pea, broadbean, lentil, soyabean and lupin. As preferred proteins, there may be mentioned, for example, the protein extract of soyabean sold by the company ISD under the name PROFAM972 or by the company LSN under the name ELESERYL, or the protein fraction of white lupin.

Preferred polymers derived from superficial body growths include any polymer obtained from body hair, nails, carapaces of insects or of crustaceans, head hair, feathers, beaks, hoofs and crests of animals. There may be mentioned, for example, chitin and its derivatives, in particular chitosan, as well as chitosan derivatives such as hydroxypropylchitosan, the succinylated derivative of chitosan, chitosan lactate, chitosan glutamate or carboxymethylchitosan succinamide, or alternatively keratin derivatives such as keratin hydrolysates and sulphonic keratins. Egg albumin may be mentioned as a preferred egg protein.

Preferred natural latexes include, for example, shellac resin, sandarac gum, dammars, elemis, copals, cellulose derivatives and mixtures of these polymers.

These natural polymers may be particulate or non-particulate.

More precisely, these natural polymers fall within the category of polysaccharides.

In general, polysaccharides may be divided into several categories.

Thus, polysaccharides that are suitable for use in the invention may be homopolysaccharides such as fructans, glucans, galactans and mannans or heteropolysaccharides such as hemicellulose.

Similarly, they may be linear polysaccharides such as pullulan or branched polysaccharides such as gum arabic and amylopectin, or mixed polysaccharides such as starch.

More particularly, the polysaccharides that are suitable for use in the invention may be distinguished according to whether or not they are starchy.

The starchy polysaccharides accordance with the invention are represented but not limited by native starches, modified starches and particulate starches.

The native starches that may be used in the present invention are more particularly macromolecules in the form of polymers consisting of elemental units which are anhydroglucose (dextrose) units, linked via a(I,4) bonds, of chemical formula (C₆HioOs)ₙ. The number of these units and their assembly make it possible to distinguish amylose, which is a molecule formed from about 600 to 1000 linearly linked glucose molecules, and amylopectin, which is a polymer that is branched every 25 glucose residues approximately ((I,6) bond). The total chain may contain between 10 000 and 100 000 glucose residues.

Starch is described in particular in Kirk-Othmer's Encyclopedia of Chemical Technology, 3rd edition, volume 21, pages 492-507, Wiley Interscience, 1983.

The relative proportions of amylose and of amylopectin, and their degree of polymerization, vary as a function of the botanical origin of the starches. On average, a sample of native starch consists of about 25 percent amylose and 75 percent amylopectin.

Occasionally, phytoglycogen is present (between 0 percent and 20 percent of starch), this molecule being an analogue of amylopectin but branched every 10 to 15 glucose residues.

Starch may be in the form of semi-crystalline granules: amylopectin is organized in leaflets, amylose forms an amorphous zone that is less well organized between the various leaflets.

Amylose self-organizes in a right-handed helix with six glucoses per turn. It dissociates into glucose which may be assimilated under the action of enzymes, amylases, all the more readily if it is in the form of amylopectin. Specifically, the helix formation does not favor the accessibility of starch to enzymes.

Starches are generally in the form of a white powder, which is insoluble in cold water, whose elemental particle size ranges from 3 to 100 microns.

By treating it with hot water, starch paste is obtained. It is used in industry for its thickening and gelling properties.

The botanical origin of the starch molecules used in the present invention may be cereals or tubers. Thus, the starches are chosen, for example, from corn starch, rice starch, cassava starch, tapioca starch, barley starch, potato starch, wheat starch, sorghum starch and pea starch.

Native starches are represented, for example, by the products sold under the names C*Amilogel™, Cargill Gel™, C* Gel™, Cargill Gum™, DryGel™ and C*Pharm Gel™ by the company Cargill, under the name Amidon de mats by the company Roquette, and under the name Pure Tapioca by the company National Starch.

The composition of the invention can comprise modified starches. The modified starches used in the composition of the invention may be modified via one or more of the following reactions: pregelatinization, degradation (acid hydrolysis, oxidation or dextrinization), substitution (esterification or etherification), crosslinking (esterification), bleaching.

The at least one natural film-former polymer is present in the composition of the invention in an amount of from about 0.1% to about 5%, preferably from about 0.01% to about 2%, more preferably from about 0.1% to about 1.5%, particularly from about 0.5% to about 1% by weight, all weights being based on the content of actives (solids) of the raw material in the total weight of the composition.

In accordance with the invention, the preferred but not limited natural film forming polymer is Hydrolyzed Corn Starch (KAMA KM13 polysaccharide resin from Lorama Group) present in the composition of the invention in an amount of from about 0.01% to about 1%, preferably from about 0.1% to about 0.5% by weight, all weights being based on the total weight of the composition.

### Polyurethane Film Forming Polymers (d)

According to the present invention, compositions comprising at least one polyurethane film forming polymer (polyurethane film former) are provided in the form of aqueous dispersion. “Aqueous polyurethane dispersion” as used herein means the aqueous polyurethane polymer dispersions disclosed in U.S. Pat. No. 7,445,770 and/or U.S. Pat. No. 7,452,770, the entire contents of both of which are hereby incorporated by reference.

More specifically, the aqueous polyurethane polymer dispersions of the present invention are preferably the reaction products of:

A) a prepolymer according to the formula:

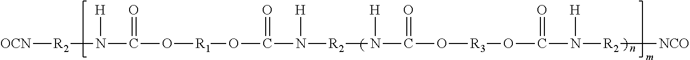

wherein R₁ represents a bivalent radical of a dihydroxyl functional compound, R₂ represents a hydrocarbon radical of an aliphatic or cycloaliphatic polyisocyanate, R₃ represents a radical of a low molecular weight diol, optionally substituted with ionic groups, n is from 0 to 5, and m is >1;
   B) at least one chain extender according to the formula: H₂N—R₄—NH₂ wherein R₄ represents an alkylene or alkylene oxide radical not substituted with ionic or potentially ionic groups; and
   C) at least one chain extender according to the formula: H₂N—R₅—NH₂ wherein R₅ represents an alkylene radical substituted with ionic or potentially ionic groups. Suitable dihydroxyl compounds for providing the bivalent radical R₁ include those having two hydroxy groups and having number average molecular weights of from about 700 to about 16,000, and preferably from about 750 to about 5000. Examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. The polyester polyols, polyether polyols and polyhydroxy polycarbonates are preferred. Mixtures of various such compounds are also within the scope of the present invention.

Suitable polyisocyanates for providing the hydrocarbon radical R₂ include organic diisocyanates having a molecular weight of from about 112 to 1,000, and preferably from about 140 to 400. Preferred diisocyanates are those represented by the general formula R₂(NCO)₂ indicated above in which R₂ represents a divalent aliphatic hydrocarbon group having from 4 to 18 carbon atoms, a divalent cycloaliphatic hydrocarbon group having from 5 to 15 carbon atoms, a divalent araliphatic hydrocarbon group having from 7 to 15 carbon atoms or a divalent aromatic hydrocarbon group having 6-15 carbon atoms. Examples of the organic diisocyanates which are suitable include tetramethylene diisocyanate, 1,6-hexamethylene diisocyanate, dodecamethylene diisocyanate, cyclohexane-1,3- and -1,4-diisocyanate, 1-isocyanato-3-isocyanatomethyl-3,5,5-trimethylcyclohexane (isophorone diisocyanate or IPDI), bis-(4-isocyanatocyclohexyl)-methane, 1,3- and 1,4-bis(isocyanatomethyl)-cyclohexane, bis-(4-isocyanato-3-methyl-cyclohexyl)-methane, isomers of toluene diisocyanate (TDI) such as 2,4-diisocyanatotoluene, 2,6-diisocyanatotoluene, mixtures of these isomers, hydrogenated TDI, 4,4′-diisocyanato diphenyl methane and its isomeric mixtures with 2,4′- and optionally 2,2′-diisocyanato diphenylmethane, and 1,5-diisocyanato naphthalene. Mixtures of diisocyanates can, of course, be used. Preferred diisocyanates are aliphatic and cycloaliphatic diisocyanates. Particularly preferred are 1,6-hexamethylene diisocyanate and isophorone diisocyanate

“Low molecular weight diols” in the context of R₃ means diols having a molecular weight from about 62 to 700, preferably 62 to 200. They may contain aliphatic, alicyclic or aromatic groups. Preferred compounds contain only aliphatic groups. The low molecular weight diols having up to about 20 carbon atoms per molecule include ethylene glycol, diethylene glycol, propane 1,2-diol, propane 1,3-diol, butane 1,4-diol, butylene 1,3-glycol, neopentyl glycol, butyl ethyl propane diol, cyclohexane diol, 1,4-cyclohexane dimethanol, hexane 1,6-diol, bisphenol A (2,2-bis(4-hydroxyphenyl)propane), hydrogenated bisphenol A (2,2-bis(4-hydroxycyclohexyl)propane), and mixtures thereof. Optionally, the low molecular weight diols may contain ionic or potentially ionic groups. Suitable lower molecular weight diols containing ionic or potentially ionic groups are those disclosed in U.S. Pat. No. 3,412,054, the contents of which is hereby incorporated by reference. Preferred compounds include dimethylol butanoic acid (DMBA), dimethylol propionic acid (DMBA) and carboxyl-containing caprolactone polyester diol. If lower molecular weight diols containing ionic or potentially ionic groups are used, they are preferably used in an amount such that <0.30 meq of COOH per gram of polyurethane in the polyurethane polymer dispersion are present

The prepolymer is chain extended using two classes of chain extenders. First, compounds having the formula: H₂N—R₄—NH₂ wherein R₄ represents an alkylene or alkylene oxide radical not substituted with ionic or potentially ionic groups. Alkylene diamines include hydrazine, ethylenediamine, propylenediamine, 1,4-butylenediamine and piperazine. The alkylene oxide diamines include 3-{2-[2-(3-aminopropoxy)ethoxy]ethoxy}propylamine (also known as dipropylamine diethyleneglycol or DPA-DEG available from Tomah Products, Milton, Wis.), 2-methyl-1,5-pentanediamine (Dytec A from DuPont), hexane diamine, isophorone diamine, and 4,4-methylenedi-(cyclohexylamine), and the DPA-series ether amines available from Tomah Products, Milton, Wis., including dipropylamine propyleneglycol, dipropylamine dipropyleneglycol, dipropylamine tripropyleneglycol, dipropylamine poly(propylene glycol), dipropylamine ethyleneglycol, dipropylamine poly(ethylene glycol), dipropylamine 1,3-propane diol, dipropylamine 2-methyl-1,3-propane diol, dipropylamine 1,4-butane diol, dipropylamine 1,3-butane diol, dipropylamine 1,6-hexane diol and dipropylamine cyclohexane-1,4-dimethanol. Mixtures of the listed diamines may also be used

The second class of chain extenders are compounds having the formula: H₂N—R₅—NH₂ wherein R₅ represents an alkylene radical substituted with ionic or potentially ionic groups. Such compounds have an ionic or potentially ionic group and two groups that are reactive with isocyanate groups. Such compounds contain two isocyanate-reactive groups and an ionic group or group capable of forming an ionic group. The ionic group or potentially ionic group can be selected from the group consisting of ternary or quaternary ammonium groups, groups convertible into such a group, a carboxyl group, a carboxylate group, a sulfonic acid group and a sulfonate group. The at least partial conversion of the groups convertible into salt groups of the type mentioned may take place before or during the mixing with water. Specific compounds include diaminosulfonates, such as for example the sodium salt of N-(2-aminoethyl)-2-aminoethane sulfonic acid (AAS) or the sodium salt of N-(2-aminoethyl)-2-aminopropionic acid

The polyurethane polymer according to the invention may also include compounds which are situated in each case at the chain ends and terminate said chains (chain terminators) as described in U.S. Pat. No. 7,445,770 and/or U.S. Pat. No. 7,452,770.

Preferably, the aqueous polyurethane polymer dispersion has a viscosity of less than 2000 mPa·s at 23 degrees centigrade, preferably less than 1500, preferably less than 1000, including all ranges and sub ranges there between. Further preferably, the aqueous polyurethane polymer dispersion has a glass transition temperature below 0 degrees centigrade

Also preferably, the aqueous polyurethane polymer dispersion has a solids (actives) content based on the weight of the dispersion of from 20 percent to 60 percent, preferably from 25 percent to 55 percent and preferably from 30 percent to 50 percent, including all ranges and sub ranges therebetween.

Also preferably, the aqueous polyurethane polymer dispersion has a glass transition temperature (Tg) that is, equal to or below −25 degrees centigrade, preferably below −35 degrees centigrade, preferably below −45 degrees centigrade.

Suitable aqueous polyurethane polymer dispersions for use in the present invention include, but are not limited to, aqueous polyurethane polymer dispersions sold under the BAYCUSAN® name by Bayer such as, for example, BAYCUSAN® C1000 (polyurethane-34), BAYCUSAN® C1001 (polyurethane 34), BAYCUSAN® C1003 (polyurethane 32), and BAYCUSAN® C1004 (polyurethane-35).

According to preferred embodiments, the at least one but not limited polyurethane dispersion used in this invention is BAYCUSAN® C1004, which contains 41% of polyurethane 35 solids(actives) and 59% of water.

According to preferred embodiments, the at least polyurethane polymer present in the composition of the present invention in an amount of solids (actives) ranging from about 1 to 15 percent by weight, more preferably from about 1.5 to about 12.5 percent by weight, more preferably from about 2 to about 10 percent by weight based on the total weight of the composition, including all ranges and sub ranges within these ranges.

### Water and Other Solvents (e)

The compositions for the invention also comprise water in an amount ranging from about 5% to about 60%, preferably from about 10% to about 55%, most typically from about 40% about 50%, including all ranges and sub ranges therebetween, by weight, relative to the total weight of the compositions.

The compositions of the invention may include additional solvents. In particular, the aqueous phase may include at least one organic solvent that is water-miscible. Non-limiting examples of suitable organic solvents include C₁₋₄ alkanols, such a sethanol and isopropanol; glycerol; glycols and glycol ethers such as 2-butoxyethanol, propylene glycol, butylene glycol, monomethyl ether of propylene glycol, monethyl ether and monomethyl ether of diethylene glycol, aromatic alcohols such as benzyl alcohol and phenoxyethanol; analogous products and mixtures of the foregoing products.

Other solvents include caprylic/capric acid triglycerides (such as those sold under the trade name Miglyol®.

In addition to water, the compositions of the invention may comprise a solvent in an amount ranging from about 0.1% to about 10%, preferably from about 3% to about 6%, including all ranges and sub ranges therebetween, by weight, relative to the total weight of the compositions.

### Pigments (f)

The cosmetic composition of the invention include at least one pigment or dyestuff. Suitable pigments/dyes include, but are not limited to, pulverulent dyestuffs, liposoluble dyes, water-soluble dyes, and pearling agents.

The pulverulent dyestuffs may, for instance, be chosen from pigments and nacres. Useful pigments include titanium dioxide, zirconium oxide, zinc oxide, cerium oxide, iron oxide, chromium oxide, manganese violet, ultramarine blue, chromium hydrate, and ferric blue. Non-limiting examples of organic pigments include carbon black, pigments of D&C type, and lakes based on cochineal carmine, barium, strontium, calcium, and aluminum.

The nacres which may be used include, for example, mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative liposoluble dyes which may be used according to the present invention include Sudan Red, DC® Red 17, DC® Green 6, beta-carotene, soybean oil, Sudan Brown, DC® Yellow 11, DC® Violet 2, DC® Orange 5, annatto, and quinolone yellow.

In an embodiment the pigment is iron oxides.

The at least one pigment/dyestuff may be present in the cosmetic composition in an amount ranging from about 5% to about 30%, more particularly from about 10% to about 25%, particularly about 20%, including all ranges and sub ranges therebetween, by weight, relative to the total weight of the compositions.

### Emollients (Optional)

The compositions of the invention include one or more emollient and/or humectants and/or moisturizers (herein “emollients”). These compounds hydrate the keratinous substrate, including the eye lids, and also provide a “wet” texture and shiny look. Emollients are known to skilled artisan. See, e.g. International Cosmetic Ingredient Dictionary and Handbook Vol. 4 (9^{th} ed. 2002), more particularly the emollients disclosed on pages 2930-2936. The disclosure of the Vol. 4, pages 2930-2936, is hereby incorporated by reference.

Without limitation, the emollients that may be used in the compositions of the invention include, for example: glycerin; glycerol; propylene glycol; butylene glycol; carnauba wax; beeswax; candelilla; ozokerite; paraffin; rice bran wax; microcrystalline wax; polyethylene wax; mineral oil; almond oil; castor oil; sesame oil; hydrogenated polyisobutene; butylene glycol dicaprylte dicaprate (commercially available from Sasol as Myglyol®); and mixtures thereof.

In a particular embodiment the emollient is butylene glycol.

The emollient is present in the composition of the invention in an amount of from about 0.1% to about 20%, preferably from about 1% to about 15%, more particularly from about 3% to about 10%, by weight, including all ranges and sub ranges therebetween, all weights being based on the total weight of the composition.

### Fillers and Pearls (Optional)

The cosmetic composition disclosed herein may also comprise at least one filler commonly used in the art in cosmetic compositions. The fillers may be lamellar or non-lamellar, inorganic or organic particles. Representative, non-limiting examples of these ingredients include mica, silica, kaolin, iron oxides, titanium dioxide, polyamide powders, polyamide powders, for instance Nylon® (Orgasol from Atochem), poly-alanine powders, polyethylene powders, tetrafluoroethylene polymer powders, for instance polytetrafluoroethylene(Teflon®), lauroyllysine, starch, boron nitride, hollow polymer microspheres such as those of polyvinylidene chloride/acrylonitrile, for instance Expancel® (Nobel Industrie), acrylic powders such as Polytrap® (Dow Corning), polymethyl methacrylate particles and silicone resin microbeads (for example, Tospearls® from Toshiba), methylsilanol/silicate crosspolymer, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example, zinc stearate, magnesium stearate, lithium stearate, zinc laurate, or magnesium myristate.

The fillers, if present, are present in amounts generally ranging from about 0.1% to about 25%, such as from about 1% to about 20% by weight, relative to the total weight of the composition, including all ranges and sub ranges therebetween.

### Further Additional Components (Optional)

The compositions of the present invention can also include any additional ingredient or additive usually used in the field of cosmetic compositions, in particular eyeliners.

For example, these may be chosen from, for example, solvents, dispersants, antioxidants (such as pentaerythrityl tetra-di-t-butyl hydroxyhydrocinnamate), preservatives (such as for example phenoxyethano, sodium dehydroacetate, disodium EDTA, caprylyl glycol, and mixtures thereof), fragrances, additional thickeners or texturizers, liquid lipids/oils, additional viscosity modifiers, additional film formers, sunscreen agents, additional pigments/colorants/dyes, silica, clays, additional humectants and moisturizing agents, additional emulsifying agents (e.g. sorbitan oleate), additional structuring agents and fillers, surfactants, shine agents, additional conditioning agents, vitamins, plant extracts, additional film-formers, coalescents/plasticizers, pH modifiers/neutralizing agents, stabilizers, and mixtures thereof. A non-exhaustive listing of such ingredients is found in U.S. Pat. No. 7,879,316, the entire content of which is hereby incorporated by reference. Additional examples of additives may be found in the International Cosmetic Ingredient Dictionary and Handbook (9^{th} ed. 2002, and subsequent editions).

### Cosmetic Methods

In an embodiment according to the invention, the compositions comprising a viscosity increasing system comprising at least one synthetic thickening polymer and at least one natural thickening polymer, at least one nonionic surfactant, at least one film forming polymer, at least one polyurethane polymer, water and pigment can provide a water-based, wax free liquid liner having one or more of the following attributes: a fresh and comfortable feel, long wear, gentle application, color intensity, and ease of removal. Accordingly, another embodiment of the invention provides a method of making up/or enhancing the appearance of eye by applying to the eyelids, topically, the composition of the present invention in a sufficient amount to make up the eyelids. The compositions may be applied to the eyelids as needed, preferably once or twice daily, and then allowed to dry before contact with clothing or other objects.

The compositions according to various exemplary embodiments of the invention may also have improved and/or increased ease of removability, relative to similar compositions that are not water based. In various embodiments, ease of removability relates to ease of removing the composition from the eyelids with warm (e.g. about 50° C. or higher) water. Optionally, corwentional cleansing agents such as soap or make-up remover may also be used.

According to the invention, the inventive compositions are characterized by good pigment suspension capability (PSC)

As presented in the examples, pigment suspension capability of the inventive compositions is at least comparable, preferably even better than commercial comparative compositions, as determined at accelerated temperature conditions (45° C.) and centrifugation tests (MethodA and B) as described later.

It has been determined that the inventive compositions show extended shelf life (reduced sedimentation and little or no phase separation) at pH from about 6 to about 8.

When the composition of the present invention is eyeliner, the composition may be packaged in an applicator product comprising a reservoir and a removable cap for closing the reservoir. The cap may, for example, form a leak-tight seal. An example of such an applicator is a pen-type applicator, such as the applicator described in U.S. Pat. Nos. 4,850,727 and 4,974,980, both of which are herein incorporated by reference.

The applicator assembly may also comprise a member for applying the composition to eyelid, wherein the applicator member allows the composition to be taken up and also allows the composition taken up to be deposited on the eyelids. This applicator member can be, for example, securely fastened to the cap for leak-tight closure of the assembly.

The applicator assembly may also comprise a draining member (or drainer) for the applicator member, the draining member possibly being securely fastened to the reservoir. The applicator member may for example, be an eyeliner brush that is well known to those skilled in the art. Such a brush for instance, comprises bristles extending outwardly from and parallel to core.

### Examples

The following Examples are intended to be non-restrictive and explanatory only, with the scope of the invention being defined by the claims.

### Method of Preparation of Inventive Compositions

The compositions of the phases noted below are identified in Table 1.
1. Phases A and B were mixed together by using a small chopping blade with holes at 70-80° C.
2. Pigments were dispersed for 1 hour within previously combined phases A and B.
3. The pre-mixed solution was cooled to 50-60° C.
4. Phase C was combined with the cooled solution.
5. The mixture was then cooled to the room temperature.
6. Then the mixture was added to phase D and homogenized completely for next 10 minutes.

TABLE 1
Inventive compositions are represented by Examples
1, 2, 3, 5 and 6. Example 4 is comparator B.

| | | | | | | | Ex. 4- |
|---|---|---|---|---|---|---|---|
| | | | | | | | compar- |
| | | Ex. 1 | Ex. 2 | Ex. 3 | Ex. 5 | Ex. 6 | ator B |
| Phase | INCI Name | (wt %)* | (wt %)* | (wt %)* | (wt %)* | (wt %)* | (wt %)* |
| A | Water | 33.05 | 34.15 | 34.05 | 44.05 | 35.7 | 49.05 |
| | Sodium Hyaluronate | 0.1 | 0.1 | 0.1 | 0.1 | | 0.1 |
| | Butylene Glycol | 2 | 2 | 2 | 2 | 2 | 2 |
| | Citric Acid | 0.05 | 0.05 | 0.05 | 0.05 | 0.05 | 0.05 |
| | Arginine | | | | | 0.05 | |
| | Hydrolyzed Corn | 0.44 | 0.44 | 0.44 | 0.44 | 0.44 | 0.44 |
| | Starch (44%) | | | | | | |
| | Water (55%) | 0.55 | 0.55 | 0.55 | 0.55 | 0.55 | 0.55 |
| | Phenoxyethanol (1%) | 0.01 | 0.01 | 0.01 | 0.01 | 0.01 | 0.01 |
| | Acrylamide/ | 0.24 | 0.24 | 0.24 | 0.24 | 0.24 | 0.24 |
| | Sodium Acryloyl- | | | | | | |
| | dimethyltaurate | | | | | | |
| | Copolymer (40%) | | | | | | |
| | Isohexadecane (21%) | 0.126 | 0.126 | 0.126 | 0.126 | 0.126 | 0.126 |
| | Polysorbate 80 (7%) | 0.042 | 0.042 | 0.042 | 0.042 | 0.042 | 0.042 |
| | Water (29.5%) | 0.177 | 0.177 | 0.177 | 0.177 | 0.177 | 0.177 |
| | Sorbitan Oleate (2.5%) | 0.015 | 0.015 | 0.015 | 0.015 | 0.015 | 0.015 |
| | Poloxamer 338 (99.95%) | 0.9995 | | | | | |
| | BHT (0.05%) | 0.0005 | | | | | |
| | Phenoxyethanol | 0.5 | 0.5 | 0.5 | 0.5 | 0.5 | 0.5 |
| | Iron Oxides | 20 | 20 | 20 | 20 | 18 | 20 |
| B | Hydroxyethylcellulose | 0.2 | 0.1 | 0.2 | 0.2 | 0.1 | 0.2 |
| | Xanthan gum | | | | | 0.1 | |
| | Ammonium | | | | | 0.368 | |
| | Acryloyldimethyl | | | | | | |
| | taurate/Steareth-8 | | | | | | |
| | Methacrylate | | | | | | |
| | Copolymer(92%) | | | | | | |
| | Tert-Butanol (3%) | | | | | 0.012 | |
| | Water (5%) | | | | | 0.02 | |
| C | Styrene/Acrylates/ | 7.868 | 7.868 | 7.868 | 7.868 | 11.802 | 7.868 |
| | Ammonium methacrylate | | | | | | |
| | Copolymer (39.34%) | | | | | | |
| | Sodium Laureth | 0.18 | 0.18 | 0.18 | 0.18 | 0.27 | 0.18 |
| | Sulfate (0.9%) | | | | | | |
| | Caprylyl Glycol (0.68%) | 0.136 | 0.136 | 0.136 | 0.136 | 0.204 | 0.136 |
| | Water (56.34%) | 11.268 | 11.268 | 11.268 | 11.268 | 16.902 | 11.268 |
| | Butylene Glycol (1.7%) | 0.34 | 0.34 | 0.34 | 0.34 | 0.51 | 0.34 |
| | Phenoxyethanol (0.83%) | 0.166 | 0.166 | 0.166 | 0.166 | 0.249 | 0.166 |
| | Tetrasodium | 0.03 | 0.03 | 0.03 | 0.03 | 0.045 | 0.03 |
| | EDTA (0.15%) | | | | | | |
| | Potassium sorbate | 0.012 | 0.012 | 0.012 | 0.012 | 0.018 | 0.012 |
| | (0.06%) | | | | | | |
| | Polyurethane-35 (41%) | 6.15 | 6.15 | 6.15 | 2.05 | 2.05 | |
| | Water (59%) | 8.85 | 8.85 | 8.85 | 2.95 | 2.95 | |
| D | Divinyldimethicone/ | 0.6 | 0.6 | 0.6 | 0.6 | 0.6 | 0.6 |
| | Dimethicone | | | | | | |
| | Copolymer (60%) | | | | | | |
| | C12-13 Pareth-23 (2.8%) | 0.028 | 0.028 | 0.028 | 0.028 | 0.028 | 0.028 |
| | C12-13 Pareth-3 (2%) | 0.02 | 0.02 | 0.02 | 0.02 | 0.02 | 0.02 |
| | Water (34.9%) | 0.349 | 0.349 | 0.349 | 0.349 | 0.349 | 0.349 |
| | Phenoxyethanol (0.3%) | 0.003 | 0.003 | 0.003 | 0.003 | 0.003 | 0.003 |
| | Alcohol Denat. | 5 | 5 | 5 | 5 | 5 | 5 |
| | Polysorbate 21 | 0.5 | 0.5 | 0.5 | 0.5 | 0.5 | 0.5 |

*In the above table, the weight percent (wt %) is shown as active components. Thus, for example for Hydrolyzed Corn Starch, 0.44% is the amount of the active.

### Evaluation of Inventive Compositions: Methods and Results

The benefits and improvements afforded by the inventive compositions were determined by comparing their pigment suspension capability (PSC) using the following tests:
1. Centrifugation at 2300 rpm (Method A) and 3000 rpm (Method B)
2. Exposure to accelerated temperature (45° C.) through 4 weeks (Method C)
3. Viscosity measurements (Method D)
4. pH measurement (Method E)
5. Gloss comparison (Method F)

The comparison of the inventive compositions was conducted against at least one of two comparator compositions:

Comparator A: commercial water based liquid eyeliner containing pigments at level greater than 15%, styrene/acrylates/ammonium methacrylate copolymer, polyester-5, pigment, propylene glycol, xanthan gum and polysorbate 80.

Comparator B: water based, wax free liquid eyeliner containing pigments at 20%, styrene/acrylates/ammonium methacrylate copolymer at about 8%, hydroxyethylcellulose at 0.2%, acrylamide/sodium/acryloyldimethyltaurate copolymer at 0.24%, polysorbate 21 at 0.5%.

In comparison to the inventive compositions, the comparators A and B differ in absence of polyurethane-35 (polyurethane polymer) and block-copolymer nonionic surfactant (poloxamer 338).

Description of Comparative Tests and their Results

### Centrifugation at 2300 Rpm (Method A)

Kimble/Chase 15 mL disposable glass centrifuge tubes were filled with 12 grams (8 cm high) of formula and they were placed in a Thermo Electron Corporation IEC CL30 Centrifuge at 2300 rpm (900 g) for 1 hour at 25° C.

### Centrifugation at 3000 Rpm (Method B)

Kimble/Chase 15 mL disposable glass centrifuge tubes were filled with 12 grams (8 cm high) of formula and they were placed in a Thermo Electron Corporation IEC CL30 Centrifuge at 3000 rpm (1200 g) for 1 hour at 25° C.

The samples tested according to Methods A and B were visually examined and physical changes in their appearance indicating composition's pigment suspension capability (phase separation, pigment agglomeration or sedimentation) were measured by using a Fisher-scientific Caliper (06-664-16).

In order to determine centrifugation stability or pigment suspension capability (PSC) of the tested compositions, the following physical characteristics were observed and recorded:
1. Observation: Syneresis (s), sedimentation (d), foaming (f), phase separation (ps), no visible separation (nvs)
2. Presence of centrifugation stability: yes, no

“Yes” was recorded if phase percent separation (% separation) was less than 5% and pellets of pigments at the bottom of the tube were not observed.

“No” was recorded if phase percent separation (% separation) was 5% or more and pellets of pigments were formed at the bottom of the tube.

If the clear pellets were formed, their height was determined by placing the test tube on its side, allowing a supernatant to reveal a lower meniscus level. The level was then marked and the height of pellets (hₚ) was measured, as well as the height of supernatant/upper phase (hₛ), the height of film former phase (hₒ) and the total height of the formula sample in the tube (h_{T}) before it was exposed to the centrifugation test.

In order to determine pigment suspension capability (PSC), relative percent separation (% separation) was calculated as the sum of the height of the film former phase and the supernatant phase (hₒ+hₛ), divided by the height of sample (h_{T}) before its exposure to the centrifugation tests. The pellets' heights were not included in the measurements.

The equation of percent separation (% separation) calculation is exemplified below:

% separation=[(hₒ+hₛ)/h_{T}]×100%

The results of the experiments conducted by the use of Method A are presented in Table 2 and 3. The results of the tests conducted according Method B are detailed in Tables 4 and 5.

TABLE 2
Results of a comparative study conducted for Comparator A and inventive compositions
Ex. 2, 3, 4 and 5. The study was conducted at the same day, using the same method.

| | | | Observations | Total height | Pigment/ | Film | Supernatant/ | |
|---|---|---|---|---|---|---|---|---|
| | | Centrifugation | (s, d, f, ps, | of filled tube | Clear pellet | former phase | upper phase | % |
| Example | Sample | stability | nvs) | (h_{T}) (mm) | (hₚ₎ (mm) | (hₒ₎ (mm) | (hₛ) (mm) | Separation |
| Comparator A | 1 | No | ps, d | 82 | 30 | 20 | 32 | 62 |
| | 2 | No | ps, d | 80 | 30 | 20 | 30 | 65 |
| Example 2 | 1 | yes | Slight to nvs | 80 | 0 | 0 | 3 | 3.8 |
| | 2 | yes | Slight to nvs | 80 | 0 | 0 | 3 | 3.8 |
| Example 3 | 1 | yes | nvs | 80 | 0 | 0 | 0 | 0 |
| | 2 | yes | nvs | 80 | 0 | 0 | 0 | 0 |
| Example 4 | 1 | no | d | 80 | 64 | 0 | 16 | 20 |
| Comparator B | 2 | no | d | 80 | 64 | 0 | 16 | 20 |
| Example 5 | 1 | no | d | 80 | 73 | 0 | 7 | 8.8 |
| | 2 | no | d | 82 | 75 | 0 | 7 | 8.5 |

TABLE 3
The inventive composition Ex. 1, was analyzed on a different day
than the compositions listed in Table 2, using the same method.

| | | | Observations | Total height | Pigment/ | Film | | |
|---|---|---|---|---|---|---|---|---|
| | | Centrifugation | (s, d, f, ps, | of filled tube | Clear pellet | former phase | upper phase | % |
| Example | Sample | stability | nvs) | (h_{T}) (mm) | (hₚ₎ (mm) | (hₒ₎ mm) | (hₛ₎ (mm) | Separation |
| Example 1 | 1 | yes | nvs | 80 | 0 | 0 | 0 | 0 |
| | 2 | yes | nvs | 80 | 0 | 0 | 0 | 0 |

Based on the results provided in Table 2 and 3 it was observed that inventive Examples 1, 2, 3, and 5 showed reductions and/or elimination of phase separation and pigment sedimentation, which proves improvement of pigment suspension capability (stability). The comparator compositions A and B, after being exposed to the same conditions as the inventive compositions had separated phases and visible pellets of pigments (pigments sedimentation).

TABLE 4
Results of a comparative study conducted for Comparator A and inventive compositions
Ex. 2, 3, 4 and 5. The study was conducted at the same day, using the same method.

| | | | Observations | Total height | Pigment/ | Film | | |
|---|---|---|---|---|---|---|---|---|
| | | Centrifugation | (s, d, f, ps, | of filled tube | Clear pellet | former phase | upper phase | % |
| Example | Sample | stability | nvs) | (h_{T}) (mm) | (hₚ₎ (mm) | (hₒ₎ (mm) | (hₛ) (mm) | Separation |
| Example 2 | 1 | no | Slight d | 80 | 0 | 0 | 6 | 7.5 |
| | 2 | no | Slight d | 80 | 0 | 0 | 5 | 6.3 |
| Example 3 | 1 | yes | Slight d | 80 | 0 | 0 | 2 | 2.5 |
| | 2 | yes | Slight d | 80 | 0 | 0 | 2 | 2.5 |
| Example 4 | 1 | no | d | 80 | 64 | 0 | 25 | 31.3 |
| Comparator B | 2 | no | d | 80 | 64 | 0 | 26 | 32.5 |
| Example 5 | 1 | no | d | 80 | 73 | 0 | 14 | 17.5 |
| | 2 | no | d | 82 | 75 | 0 | 15 | 18.8 |

TABLE 5
The inventive composition Ex. 1, was analyzed on a different day
than the compositions listed in Table 4, using the same method.

| | | | Observations | Total height | Pigment/ | Film | | |
|---|---|---|---|---|---|---|---|---|
| | | Centrifugation | (s, d, f, ps, | of filled tube | Clear pellet | former phase | upper phase | % |
| Example | Trial | stability | nvs) | (h_{T}) (mm) | (hₚ₎ (mm) | (hₒ₎ (mm) | (hₛ₎ (mm) | Separation |
| Example 1 | 1 | yes | Slight d | 80 | 0 | 0 | 2 | 2.5 |
| | 2 | yes | Slight d | 80 | 0 | 0 | 2 | 2.5 |

Based on the results provided in Table 4 and 5 it was observed that the inventive Examples 1, 2, 3 and 5 were characterized by reduction of visible phase separation and pigment sedimentation, which shows improvement of pigment suspension capability (PSC) of the inventive compositions. The comparator composition B after being exposed to the same conditions had separated phases and visible pellets of pigments (pigments sedimentation).

### Exposure to Accelerated Temperature (Method C)

The tested compositions were exposed to temperature of 45° C. for the period of 4 weeks.

The tested compositions were examined for the following characteristics:
1. Presence of stability: yes, no
2. Viscosity measurements
3. pH measurements
4. visual observation of: syneresis (s), sedimentation (d), foaming (f), phase separation (ps), no visible separation (nvs)

### Viscosity (Method D)

The viscosity of the liquid eyeliners were measured at 25° C. with Rheomat 180 viscometer at 200 rpm (revolutions per minute) using a No. 3 or 4 spindle (hereinafter, “Method D”). The viscosity values were taken 10 minutes after switching on the rotation of the spindle and the values were reported in Pa*s.

### Method of pH Measurements (Method E)

pH values were measured using a Denver Scientific Ultrabasic pH Meter with a 3 mol/L KCl probe.

The outcomes of accelerated temperature tests are presented in Table 6.

TABLE 6
Results of compositions' exposure to accelerated temperature test.

| | | | Specifications | | |
|---|---|---|---|---|---|
| | Time | | Viscosity | | |
| | point of | Stability | (spindle | | Observations |
| | samples | (yes or | number 3 | | (s, d, f, ps, |
| Examples | examinations | no) | or 4)* Pa · s | pH | n.s., g, nvs) |
| Comparator | initial | no | — | — | ps, d |
| A | | | | | |
| Example 1 | initial | yes | 0.9(3) | 7.52 | nvs |
| | 4 weeks, | yes | 0.9(3) | 7.53 | nvs |
| | 45° C. | | | | |
| Example 2 | initial | yes | 0.69(3) | 7.69 | nvs |
| | 4 weeks, | yes | 0.73(3) | 7.71 | nvs |
| | 45° C. | | | | |
| Example 3 | initial | yes | 0.94(3) | 7.57 | nvs |
| | 4 weeks, | yes | 1.03(3) | 7.68 | nvs |
| | 45° C. | | | | |
| Example 4 | initial | yes | 0.69(3) | 7.62 | s |
| Comparator | 4 weeks, | no | n.d. | n.d. | Ps, d |
| B | 45° C. | | | | |

*Measured with Method D

As the above results show, visible separation or sedimentation was not observed in Examples 1-3 after 4 weeks exposure to 45° C. In contrast, comparative Example 4 which contained neither Poloxamer-338 nor Polyurethane-35, clearly showed phase separation and sedimentation of pigments after 4 weeks exposure to 45° C., which proves deficiency in pigment suspension capability (stability). There were no changes in pH values during course of experiments in Examples 1-4.

### Gloss Test (Method F)

### Procedure for Test:

The gloss of the inventive compositions as well as of Comparator A was measured using a BYK Gardner micro glossmeter and following the ASTM Standard Test Method for determining Gloss as described at:

http://www.opbpak.com/ASTM/D2457-03.pdf, or
http://www.astm.org/Standards/D523.htm.

The results of this test are reported below in Table 7.

Gloss measurements were taken from 1.5 mil drawdowns of formulas on Laneta Black and White draw down cards. “Drawdown” is a term of art in the cosmetic industry. In this procedure, the compositions were scooped and spread evenly on the cards using a metal “drawdown” bar. Once the films dried, the shine of the resulting films was measured on a BYK Gardner micro gloss meter. The measurements are reported in gloss units (GU) which represent the ratio of reflected to incident light of the films compared to that for a standard. Low gloss is a GU measurement less than 10 at a measurement angle of 85 degrees. The results in Table 7 are an average of three trials (n=3).

TABLE 7
Results of Gloss Test

| | GU Values at 60° average | GU Values at 85° average |
|---|---|---|
| Example | (st. dev), n = 3 | (st. dev), n = 3 |
| Comparator A | Could not obtain drawdown | Could not obtain drawdown |
| | due to formula instability | due to formula instability |
| Example 1 | 2.8 (0.0) | 26.4 (0.3) |
| Example 2 | 0.5 (0.0) | 14.2 (0.6) |
| Example 3 | 0.5 (0.0) | 15.1 (0.5) |
| Example 4 | 0.3 (0.0) | 9.6 (0.5) |
| comparator B | | |

Table 7 above, shows that the inventive compositions of Examples 1, 2 and 3 yielded a cosmetic product that was not glossy (had gloss values less than 10 GU). This is desirable for eyeliners as consumers prefer non-shiny, natural looking eyeliners.

What is claimed is:

1. A water-based, wax free liquid cosmetic composition comprising:
(a) a viscosity increasing system comprising (I) at least one synthetic thickening polymer; and (II) at least one natural thickening polymer;
(b) at least one nonionic surfactant;
(c) at least one film forming polymer;
(d) at least one polyurethane polymer;
(e) water; and
(f) a pigment;
wherein the composition has a viscosity of less than about 7 Pa·s.

2. The composition of claim 1 wherein the ratio of the at least one synthetic thickening polymer (a)(I) to the at least one natural thickening polymer (a)(II) is less than or equal to 4:1.

3. The composition of claim 2 wherein the viscosity increasing system is present in an amount of from about 0.01% to about 1.5%, by weight, relative to the total weight of the composition.

4. The composition of claim 3 wherein the viscosity increasing system (a) comprises (I) from about 0.01% to about 1.0% by weight of at least one synthetic thickening polymer; and (II) from about 0.01% to about 0.5% by weight of at least one natural thickening polymer, by weight relative to the total weight of the composition.

5. The composition of claim 4 wherein the at least one nonionic surfactant (b) is present in an amount of from about 0.1% to about 5%, by weight, relative to the total weight of the composition.

6. The composition of claim 5 wherein the at least one film forming polymer (c) is present in an amount of from about 0.01% to about 20%, by weight, relative to the total weight of the composition.

7. The composition of claim 6 wherein at least one polyurethane polymer (d) is present in an amount of from about 1.0% to about 15%, by weight, relative to the total weight of the composition.

8. The composition of claim 7 wherein the water (e) is present in an amount of from about 5% to about 60%, by weight, relative to the total weight of the composition.

9. The composition of claim 4 wherein the ratio of the at least one synthetic thickening polymer (a)(I) to the at least one natural thickening polymer (a)(II) is from about 1:1 to about 4:1.

10. The composition of claim 5 wherein the at least one nonionic surfactant (b) is selected from: esters and ethers of monosacharides, oxyethylenated and oxypropylenaled ethers, esters of fatty acids and polyethylene glycols, esters of fatty acids and sorbitol ethers, carbohydrate based esters, block-copolymer surfactants and mixtures thereof.

11. The composition of claim 10 wherein the nonionic surfactant (b) is the mixture of carbohydrate based ester surfactants and block-copolymer surfactants.

12. The composition of claim 11 wherein the ratio of carbohydrate based ester surfactants to block-copolymer surfactants is about 1:2.

13. The composition of claim 1 wherein the ratio of the at least one polyurethane polymer (d) to the at least one natural thickening polymer (a)(II) is from about 10:1 to about 60:1.

14. The composition of claim 1 comprising:
(a) from about 0.01% to about 1% by weight of at least one synthetic thickening polymer (a)(I); and from about 0.01% to about 0.5% by weight of at least one natural thickening polymer (a)(II);
(b) from about 0.1% to about 5% by weight of at least one nonionic surfactant;
(c) from about 0.01% to about 20% by weight of at least one film forming polymer;
(d) from about 1% to about 15% by weight of at least one polyurethane polymer;
(e) from about 5% to about 60% by weight of water; and
(f) from about 5% to about 30% by weight of at least one pigment.

15. A water-based, wax free liquid cosmetic composition comprising:
(a) a viscosity increasing system comprising (I) at least one synthetic thickening polymer; and (II) at least one natural thickening polymer;
(b) at least one nonionic surfactant selected from: esters and ethers of monosacharides, oxyethylenated and oxypropylenaled ethers, esters of fatty acids and polyethylene glycols, esters of fatty acids and sorbitol ethers, carbohydrate based esters, block-copolymer surfactants and mixtures thereof;
(c) at least one film forming polymer;
(d) at least one polyurethane polymer;
(e) water; and
(f) a pigment;
wherein the ratio of the at least one polyurethane polymer (d) to the at least one natural thickening polymer (a) (II) is greater than 10:1; and the ratio of the at least one synthetic thickening polymer (a)(I) to the at least one natural thickening polymer (a)(II) is from about 1:1 to about 4:1.

16. The composition of claim 15 wherein the nonionic surfactant (b) is the mixture of carbohydrate based ester surfactants and block-copolymer surfactants.

17. The composition of claim 16 wherein the at least one natural thickening polymer (a)(II) is present in an amount of from 0.01% to about 0.5% by weight, relative to the total weight of the composition.

18. The composition of claim 17 wherein the at least one natural thickening polymer (a)(II) is present in an amount of about 0.2% by weight, relative to the total weight of the composition.

19. The composition of claim 18 wherein the at least one nonionic surfactant (b) is present in an amount of from about 0.1% to about 5% by weight, relative to the total weight of the composition.

20. The composition of claim 19 wherein the at least one film forming polymer (c) is present in an amount from about 0.01% to about 20% by weight, by weight, relative to the total weight of the composition.

21. The composition of claim 20 wherein the at least one polyurethane polymer (d) is present in an amount of from about 1% by to about 15% by weight, relative to the total weight of the composition.

22. The composition of claim 21 wherein the water (e) is present in an amount of from about 5% to about 60% by weight, relative to the total weight of the composition.

23. The composition of claim 15 wherein the ratio of the at least one polyurethane polymer (d) to the at least one natural thickening polymer (a) (II) is from about 10:1 to about 60:1.

24. The composition of claim 23 having a viscosity of less than 7 Pa·s.

25. A kit for making up the eyes comprising a pen-type applicator containing the composition of claim 1.

26. A method of making up or enhancing the appearance of the eye by applying to the eyelids, topically, a composition according to claim 1.

27. Method of making the water-based, wax free liquid cosmetic composition comprising:
(a) from about 0.01% to about 1% by weight of at least one synthetic thickening polymer (a)(I); and from about 0.01% to about 0.5% by weight of at least one natural thickening polymer (a)(II);
(b) from about 0.1% to about 5% by weight of at least one nonionic surfactant;
(c) from about 0.01% to about 20% by weight of at least one film forming polymer;
(d) from about 1% to about 15% by weight of at least one polyurethane polymer;
(e) from about 5% to about 60% by weight of water; and
(f) from about 5% to about 30% by weight of at least one pigment;
wherein the composition has a viscosity of less than about 7 Pa·s.
